(19) United States
(12) Patent Application Publication
(10) Pub. No.: US 2025/0018013 A1
(43) Pub. Date: Jan. 16, 2025

(54) COMPOSITION INCLUDING MICROBIAL HEMOPROTEIN EXTRACT FOR IMPROVING INTESTINAL FUNCTION AND OBESITY

(71) Applicant: HEMOLAB LTD. CO., Seoul (KR)

(72) Inventors: Byung Ah KIM, Seoul (KR); Seung Ki LEE, Anyang-si (KR); Kyung Hoon PARK, Seoul (KR)

(73) Assignee: HEMOLAB LTD. CO., Seoul (KR)

(21) Appl. No.: 18/713,167

(22) PCT Filed: Nov. 16, 2022
(86) PCT No.: PCT/KR2022/018072

§ 371 Date: May 23, 2024

(30) Foreign Application Priority Data
Nov. 25, 2021 (KR) 10-2021-0164874
Jun. 3, 2022 (KR) 10-2022-0068513

(51) Int. Cl. A61K 38/42; A61P 3/04; C12N 1/20; C12R 1/15

(52) U.S. Cl. CPC A61K 38/42; A61P 3/04; C12N 1/205; C12R 2001/15

(57) ABSTRACT

The present disclosure relates to use of microorganisms with high hemoprotein content, or culture thereof for gut health and weight control, and particularly to a composition for activating and improving intestinal flora and controlling body weight, comprising a microbial hemoprotein extract obtained from culture of microorganisms with a high hemoprotein content.

### TECHNICAL FIELD

The present disclosure relates to use of microorganisms with high hemeprotein (also referred to as hemoprotein) content, culture thereof, or hemoprotein extract thereof for improving gut health and obesity, and in particular, relates to a composition for activating and improving intestinal flora and alleviating obesity-related conditions, comprising a microbial hemeprotein extract obtained by culturing microorganisms with a high hemeprotein content.

### BACKGROUND ART

Obesity does not simply mean weight gain, but refers to a condition in which excessive body fat is accumulated in the body. Obesity is on the rise worldwide, and in addition to aesthetic problems, obesity may also become a primary cause for diseases such as high blood pressure, diabetes, cardiovascular disease, breast cancer, and colorectal cancer, and lead to complications. The World Health Organization (WHO) views obesity as a disease that requires treatment.

There are many factors that contribute to obesity, including genetic factors such as problems with leptin, an appetite-suppressing protein, mental factors such as eating to satisfy psychological/physiological needs, social factors such as overconsumption of food and westernization, and lifestyle factors such as lack of exercise.

The large intestine, which is the last part of the digestive system, has not received much attention other than its role as a tube within animals, but recently, when it was found to contain a large number of neurons and produce 95% or more of serotonin, its importance has increased to a point where it is referred to as a second brain. Length and surface area of a human large intestine is known to be 1.5 m and 300 m², respectively, and the total number of microbial communities (flora) present in a human colon is about 10¹³⁻¹⁴ or more. The human intestinal flora consists of several phyla, including Firmicutes, Bacteroidetes, Proteobacteria, and Actinobacteria, of which at least 400 species are known. In addition, the proportion is high enough to account for at least 30% of fecal solids, and the activity of these intestinal flora is known to have diverse influences in the human body including immunity, medicinal efficacy, and digestive metabolism. In particular, intestinal flora is attracting attention as it is known to play an anti-obesity role due to the association between lactobacillus species and fat metabolism.

Heme iron is a prosthetic group and is present in nature in various types of hemoproteins (hemoprotein=heme−conjugated protein) bound to proteins, and plays essential roles in the energy production process of living organisms, such as the transfer of oxygen molecules (O₂) (hemoglobin in erythrocytes, myoglobin in muscles, and leghemoglobin in legumes), transfer of electrons (various types of cytochromes, reductase, oxygenase, etc.), and removal of toxic oxygen (catalase, peroxidase, etc.), and in the removal of reactive oxygen species (ROS). However, because lactic acid bacteria do not have a pathway to biosynthesize heme iron, they use fermentation metabolism and have the disadvantage of being vulnerable to oxygen. In natural conditions, lactic acid bacteria import heme iron from outside and use the heme iron as the hemoprotein they need. Therefore, the supply of hemoprotein including heme iron from the outside may increase the survival of lactic acid bacteria and may serve as probiotics in the intestinal environment. However, animal hemoprotein derived from animal sources is not desirable in terms of weight management.

Korean Patent Application Publication No. 2018-0049612 relates to a composition for promoting the growth of lactobacillus or strengthening preservation, comprising microbial hemoprotein, and discloses that the microbial hemoprotein extract increases the production of lactobacillus biomass and preservative strength thereof by increasing viability. However, it does not disclose or suggest probiotic activity or weight control by microbial hemoprotein extract.

The inventors of the present disclosure conducted research on a method to improve gut health and the prevention or treatment of obesity by using lactic acid bacteria, and found that heme iron extract obtained by culturing microorganisms has excellent effects on probiotic activity and weight control, and thus completed the present disclosure.

### DISCLOSURE

### Technical Problem

The present disclosure is to provide a composition for improving intestinal function and obesity, comprising a microbial hemoprotein extract.

The present disclosure is also to provide a feed additive for improving intestinal function and obesity, comprising a microbial hemoprotein extract.

### Technical Solution

One aspect of the present disclosure provides a composition for improving intestinal function and obesity, comprising a microbial hemoprotein extract.

As used herein, the term “hemoprotein” or “hemeprotein” refers to a protein to which heme iron is conjugated, which may be produced by heme-producing microorganisms and accumulated inside the microorganism or be released to the outside and exist in the culture broth, and may be the microorganism itself, its culture broth, or a microbial protein (single cell protein) or extract thereof with a high content of hemoprotein extracted therefrom. Hemoprotein includes heme iron produced by a microorganism, or a protein or microbial biomass including heme iron, and the term is used interchangeably with “microbial hemoprotein” or “microbial hemoprotein extract” because it is derived from microorganisms.

As used herein, the term “heme iron” refers to a porphyrin ring complexed with an iron and is used interchangeably with heme. Heme iron is a molecule found in all living organisms with respiratory metabolism, including animals, plants, and microorganisms, and is a general term for coordinate iron porphyrin-based molecules having various side chains.

As used herein, the terms “hemoprotein extract”, “microorganism-derived hemoprotein extract”, or “microbial hemoprotein” refer to culture or culture broth of a microorganism with high hemoprotein content, an extract including hemoprotein isolated or extracted therefrom, hemoprotein or heme iron itself, or a composition including them. As used herein, the terms hemoprotein extract and hemoprotein are used interchangeably.

As used herein, the term “microorganism with high hemoprotein content” refers to a microorganism that accumulates hemoprotein inside and outside thereof while growing and thus has a higher hemoprotein content compared to microorganisms with no or low heme iron production ability. As used herein, “microorganism with high hemoprotein content” is used interchangeably with “microorganism with heme iron production ability”. The microorganism with high hemoprotein content may have been selected through adaptive evolution to select a microorganism with high growth rate and correspondingly high biosynthetic capacity without any genetic manipulation. For example, the microorganism may be Corynebacterium glutamicum HemoP1 disclosed in Korean Patent No. 210764 or Klebsiella variicola HemoC1 disclosed in Korean Patent No. 2118083.

As used herein, the term “intestinal function” or “probiotic activity” refers to improving the overall function of the intestine or gut health by improving the intestinal flora. Improving the intestinal flora refers to increasing the type and number of beneficial bacteria, such as lactic acid bacteria and reducing the type and number of harmful bacteria in the intestinal flora.

As used herein, the term “weight control” or “weight loss support” refers to supporting or assisting in restoring and maintaining a normal body weight through weight loss or inhibition of weight gain, and may include the treatment or prevention of obesity by reducing body fat, or inhibiting accumulation of body fat.

As used herein, the terms “controlling obesity” or “improving obesity” refer to controlling or reducing weight gain or body fat accumulation that causes or worsens obesity.

In an embodiment of the present disclosure, the hemoprotein extract may be in a form of a microbial culture broth obtained by culturing a heme iron-producing microorganism, a microbial biomass isolated therefrom, or a hemoprotein isolated and purified therefrom.

In an embodiment of the present disclosure, the heme iron-producing microorganism or microorganism with a high content of hemoprotein may be a non-genetically modified microorganism selected through adaptive evolution for growth rate and heme iron production capacity to have a high hemoprotein content compared to the parent strain, without genetic manipulation. For example, it may be Corynebacterium glutamicum HemoP1 disclosed in Korean Patent No. 210764 or Klebsiella variicola HemoC1 disclosed in Korean Patent No. 2118083.

In an embodiment of the present disclosure, the microorganism with high hemoprotein content may be a microorganism with naturally increased activity or expression level of enzymes involved in energy metabolism or reactive oxygen species detoxification, isolated from nature without artificial genetic manipulation, for example, a microorganism of the genus Corynebacterium.

In an embodiment of the present disclosure, the microorganism with high hemoprotein content may be Corynebacterium glutamicum, which is derived from healthy intestinal flora and includes a higher hemoprotein content than general microorganisms due to its high energy metabolism, fast growth rate, high resistance to oxidative stress, and red color when disrupted.

In an embodiment of the present disclosure, the Corynebacterium glutamicum may be Corynebacterium glutamicum HemoP1 as disclosed in Korean Patent No. 210764.

As used herein, the term “parent strain” refers to an original strain prior to being modified by recombinant technique, mutagenesis, or adaptive evolution.

In an embodiment of the present disclosure, the hemoprotein extract may be in the form of a culture or culture broth of Corynebacterium glutamicum, a dried culture or culture broth thereof, an extract of the culture, or a purified hemoprotein extract from the culture. The culture broth obtained by culturing of (Corynebacterium glutamicum include hemoprotein produced by Corynebacterium glutamicum and may therefore be used as a source of hemoprotein or heme iron per se, or as a hemoprotein extract or heme iron extract obtained by further extracting hemoprotein or heme iron from the culture, or as hemoprotein or heme iron obtained by further purifying the extracted hemoprotein or heme iron.

In an embodiment of the present disclosure, the hemoprotein extract may be in the form of a culture or culture broth of Klebsiella variicola, a dried culture or culture broth thereof, an extract of the culture, or a purified hemoprotein extract from the culture. The culture broth obtained by culturing of Klebsiella variicola include hemoprotein produced by Klebsiella variicola and may therefore be used as a source of hemoprotein or heme iron per se, or as a hemoprotein extract or heme iron extract obtained by further extracting hemoprotein or heme iron from the culture, or as hemoprotein or heme iron obtained by further purifying the extracted hemoprotein or heme iron.

In an embodiment of the present disclosure, the composition may increase the proportion of Firmicutes in the intestinal flora. Firmicutes is a group to which lactic acid bacteria belong, and an increase in the proportion of intestinal flora belonging to Firmicutes leads to improvement in intestinal function.

In an embodiment of the present disclosure, the hemoprotein extract may be obtained by recovering microbial biomass from the microbial culture broth, resuspending and disrupting the recovered microbial biomass, and centrifuging or drying the disrupted suspension.

In an embodiment of the present disclosure, the culturing of the microorganism with high hemoprotein content may be performed using a medium known in the art to which the present disclosure belongs. Culture methods and conditions may be selected by those skilled in the art to which the present disclosure belongs.

In an embodiment of the present disclosure, the composition may help to control body weight by reducing the ratio of adipose tissue weight to body weight.

In an embodiment of the present disclosure, the composition may further include an active ingredient for improving intestinal function, weight control, or improving obesity.

In an embodiment of the present disclosure, the composition may be a feed raw material, food raw material, or health functional food, and may be used in the form of powder, granule, pill, tablet, capsule, food, beverage, or the like.

In an embodiment of the present disclosure, the content of hemoprotein extract in the composition may be between 0.01 wt % and 70 wt % based on the total weight of the composition.

In an embodiment of the present disclosure, the composition may be administered in an amount of 0.0001 mg/kg to 100 mg/kg of hemoprotein extract per day, preferably 0.001 mg/kg to 100 mg/kg. The preferred dose of the microbial hemoprotein extract of the present disclosure may be appropriately selected by a person skilled in the art depending on the condition and weight of the individual, the degree of obesity, the formulation of the composition, the route and duration of administration. The dose may be administered once a day or in several divided doses.

Another aspect of the present disclosure provides a feed additive for improving intestinal function and obesity, comprising a hemoprotein extract.

As used herein, the term “feed additive” refers to a substance added to feed for the purpose of strengthening specific functionality or nutrition, etc., and may be administered in combination with feed, or administered alone.

In an embodiment of the present disclosure, the hemoprotein extract may be a microbial culture broth obtained by culturing a microorganism with a high hemoprotein content or a hemoprotein extract obtained therefrom.

In an embodiment of the present disclosure, the feed additive may be to improve feed conversion ratio (FCR).

As used herein, the term “feed conversion ratio (FCR)”, a measure of the nutritional value of feed, refers to the amount of feed (kg) required to increase 1 kg of body weight of a subject animal, and is calculated as feed intake/weight gain. The term is used interchangeably with feed efficiency.

The feed additive according to an embodiment of the present disclosure may be manufactured in the form of powder or granules, and, if necessary, may additionally include one or more of natural antioxidants such as organic acids including citric acid, fumaric acid, adipic acid, lactic acid, and malic acid, phosphates including sodium phosphate, potassium phosphate, acid pyrophosphate, and polyphosphate, polyphenols, catechin, alpha-tocopherol, rosemary extract, vitamin C, green tea extract, licorice extract, chitosan, tannic acid, and phytic acid.

Feed additives according to an embodiment of the present disclosure may further include grains, for example milled or shredded wheat, oats, barley, corn, and rice; vegetable protein feeds, for example feeds based primarily on rapeseed, soybean, and sunflower; animal protein feeds, for example blood meal, meat meal, bone meal, and fish meal; sugars and dairy products, for example dry ingredients including various milk powder and whey powders; and may further include nutritional supplements, digestion and absorption enhancers, growth promoters, and the like.

The feed additive according to an embodiment of the present disclosure may contain preservatives, stabilizers, wetting or emulsifying agents, solution boosts, and the like.

### Advantageous Effects

A composition and feed additive for improving intestinal function and obesity comprising a hemoprotein extract according to an embodiment of the present disclosure may improve intestinal flora and thus lead to gut health and reduction in weight gain to effectively ameliorate obesity by utilizing hemoprotein obtained safely and economically by culturing of a heme iron-producing microorganism.

### DESCRIPTION OF DRAWINGS

FIG. 1 is a graph showing comparison of the effect of a hemoprotein extract according to an embodiment of the present disclosure on intestinal flora of mice.

FIG. 2 shows a normal control group (N), an obese control group (C), a hemoprotein extract 0.5 g/kg mixed feeding group (SL), and a hemoprotein extract 5 g/kg mixed feeding group (HL) mice after completion of two weeks of feeding with a diet including a hemoprotein extract according to an embodiment of the present disclosure.

FIG. 3A-3D shows the effect of a hemoprotein extract according to an embodiment of the present disclosure on adipose tissue weight of mice. FIG. 3A to 3D represent peri-epididymal fat, peri-renal fat, retroperitoneal fat, and mesenteric adipose tissue weight, respectively.

FIG. 4A-4D shows the effect of a hemoprotein extract according to an embodiment of the present disclosure on the ratio of adipose tissue weight to body weight in a mouse subject. FIG. 4A to 4D represent a ratio of peri-epididymal fat, peri-renal fat, retroperitoneal fat, and mesenteric adipose tissue weight to body weight, respectively.

FIG. 5 is a graph comparing the effect of a hemoprotein extract according to an embodiment of the present disclosure on intestinal flora of a dog.

### MODE FOR INVENTION

### Example 1. Preparation of Microbial Hemoprotein Extract

A single colony of Corynebacterium glutamicum HemoP1 (Korean Patent No. 10-2210764) or Klebsiella variicola HemoC1 (Korean Patent No. 10-2118083) strain, which has been confirmed to have high microbial hemoprotein content, was inoculated into 15 mL of YS medium (yeast extract 0.5% w/v, soytone 1% w/v, and glucose 1% w/v) in a test tube and incubated with shaking at 250 rpm at 30° C. for 16 hours before transferring to a 5 L jar fermenter containing 3 L of the same medium. After culturing at 30° C. with aeration and stirring at 0.5 vvm and 250 rpm for 48 hours, the obtained culture was centrifuged at 3,000 g for 15 minutes at 4° C. to recover the microbial biomass and washed twice with distilled water. The recovered microbial biomass was suspended in 100 mL of distilled water and then subjected to a high pressure homogenizer (EmulfsiFlex-C3, Sonic corp. Stratford, CT, USA) operated at 15,000 psi for three repeated blasts to disrupt the cells and release hemoprotein. After disruption, the resulting suspension was dried in a 105° C. oven for 24 hours to obtain microbial hemoprotein.

### Example 2. Weight Control and Intestinal Function in Mice Fed with Microbial Hemoprotein

Effects on body weight change and intestinal function were investigated using a diet including a microbial hemoprotein obtained in Example 1.

Specifically, 20 male 12-week-old ICR mice (Hana Biotech, Ansan, Gyeonggi-do, Korea) were used after they were confirmed as healthy following inspection and acclimation for 7 days at Dongnam Chemical Research Institute Animal Center (Animal Facility Registration Certificate No. 412). Within the cage, individuals were identified by ear punching, and grouped so that the mean and standard deviation of body weight were uniform among groups. A general feed for laboratory animals (Sam Taco Bio Korea, Osan-si, Gyeonggi-do, Korea) was fed ad libitum. Animals were divided into four groups: general feed powder (MF), general feed+1% (w/w) lactic acid bacteria powder (Harucare, CJ CheilJedang) mixed feed (MF+LAB 1% (w/w)), general feed+1% (w/w) lactic acid bacteria powder+1% (w/w) HemoP1 hemoprotein extract powder mixed feed (MF+LAB+HP(P) 1% (w/w)), and general feed+1% (w/w) lactic acid bacteria powder+1% (w/w) HemoC1 hemoprotein extract powder mixed feed (MF+LAB+HP(C) 1% (w/w)), and were fed for 5 days. Body weight was measured before and after the start of feeding, and fecal samples were collected. Statistical tests were performed using the StatView statistical program with p<0.05 or less, and p<0.001 considered statistically significant. For each item, statistical significance was verified using t-test one-way ANOVA.

Table 1 below shows the results of body weight measurement by diet. Body weight is expressed as the mean and standard deviation for each group (n=5).

TABLE 1

| | | Body weight gain/ |
|---|---|---|
| | Body weight after 5 | loss after 5 days |
| normal diet | days of feeding (g) | of feeding (g) |
| MF | 32.103 ± 0.535 | +0.352 |
| MF + LAB 1%, live | 31.632 ± 1.164 | +0.119 |
| bacteria | | |
| MF + LAB + HP(P) 1% | 31.490 ± 0.941 | −0.261 |
| MF + LAB + HP(C) 1% | 31.781 ± 0.889 | −0.030 |

*Initial average body weight of 20 12-week-old male mice: 31.751 ± 0.868

As shown in Table 1, when general feed was fed, the body weight gain after 5 days of feeding was 0.352 g and when only lactic acid bacteria were fed in addition to the general powder (MF+LAB), it was 0.119 g. When the hemoprotein extract of the HemoP1 strain (MF+LAB+HP(P)) or the hemoprotein extract of the HemoC1 strain (MF+LAB+HP(C)) was added together with the lactic acid bacteria, the body weight was reduced by 0.261 g and 0.030 g, respectively. When observed with the naked eye, the feces collected after 5 days of feeding were all of normal shape and form, but the feces collected from the mouse cage belonging to the group (MF+LAB) in which only lactic acid bacteria were added to the general feed were found slightly moister.

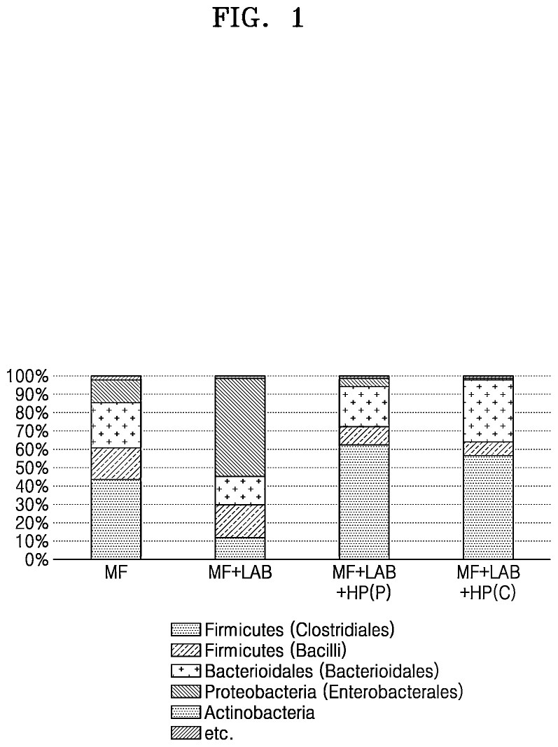

In addition, to identify the effect on the intestinal function, the distribution of intestinal flora in fecal samples collected 5 days after the start of feeding was analyzed based on 16S rRNA sequence (Cheonlab Microbiome Analysis Laboratory, Seoul, Korea). The results are shown in FIG. 1.

In the feces of the group fed 1% lactic acid bacteria powder together with general feed (MF+LAB) compared to the group fed general feed (MF), the distribution of intestinal flora showed significant reduction from 61% to 29% in the phylum Firmicutes (Clostridiales), to which lactic acid bacteria belong, and significant increase from 8% to 55% in the phylum Proteobacteria (Enterobateriales), which includes E. coli, etc. These results suggest that the rapid increase in the proportion of proteobacteria with high sugar utilization rate in the intestinal flora is attributable to the sugar component included in the lactic acid bacteria powder, rather than the intestinal colonization of lactic acid bacteria contained in commercial lactic acid bacteria powder, which was consistent with the increase in fecal moisture content observed by the naked eye. On the other hand, it was found that when microbial hemoprotein (HP) derived from HemoP1 or HemoC1 was fed together with commercial lactic acid bacteria powder, the proportion of the Firmicutes was 72% and 63%, respectively, which were all increased compared to that in the general feed (MF) group or the MF and lactic acid bacteria powder group.

### Example 3. Effect of Microbial Hemoprotein Extract on Body Weight Control and Intestinal Function in Chicken

In the present Example, the microbial hemoprotein obtained in Example 1 was supplied to broiler chickens and laying hens as a feed additive, and the resulting effects on body weight control and intestinal function were investigated.

### 3-1. 1st Broiler Chicken

A dietary test was conducted for a total of 32 days using 160 1-day-old broiler chickens (ROSS 308) as experimental animals to confirm the effect of microbial hemoprotein on body weight control and intestinal function (New Materials Research Institute Test Farm, Deokso, Gyeonggi-do, Korea). 4 groups with 10 chickens per group, general feed (BD, basal diet, JoongAng Livestock broiler feed), general feed and 1 ppm of hemoP1 hemoprotein (BD+hemoprotein 1 ppm), general feed and 10 ppm of hemoP1 hemoprotein (BD+hemoprotein 10 ppm), and general feed and organic iron (BTRAX M-Fe) 50 ppm (BD+organic iron (BTRAX M-Fe) 50 ppm) were formed and the dietary test was conducted in four replicates. During this period, the body weight gain and feed intake of the broilers were measured, and a cecum was excised and plated on NB and MRS medium to measure the CFU of lactic acid bacteria.

TABLE 2

| | | | Lactic acid | | |
|---|---|---|---|---|---|
| | FCR | FCR | bacteria in | | |
| | (feed intake/ | ratio | the cecum | Body weight | Body weight |
| Diet | weight gain) | (%) | (10⁸ cfu/g) | gain (g) | gain rate (%) |
| BD | 1.42 ± 0.08 | 100.0 | 3.7 | 1579.3 ± 21.4 | 100 |
| BD + Hemoprotein1 | 1.33 ± 0.06 | 106.8 | 6.9 | 1553.9 ± 31.0 | 97.1 |
| ppm | | | | | |
| BD + Hemoprotein | 1.34 ± 0.04 | 106.0 | 8.8 | 1559.0 ± 43.6 | 98.7 |
| 10 ppm | | | | | |
| BD + Organic | 1.32 ± 0.05 | 107.6 | 2.1 | 1582.6 ± 44.1 | 100.2 |
| Iron (BTRAX | | | | | |
| M-Fe) 50 ppm | | | | | |

As shown in Table 2, broilers fed 1 ppm and 10 ppm of hemoprotein along with general feed (BD) gained 2.9% and 1.3% less body weight, respectively, than broilers fed general feed (BD) alone. As a negative control, broilers fed with a diet including organic iron (BD plus 50 ppm organic iron (BTRAX M-Fe)) showed little change in body weight gain compared to the general feed group. The CFU of lactic acid bacteria in the cecum excised after slaughter of the broilers fed 1 ppm and 10 ppm of hemoprotein along with general feed (BD) also increased by 3.2×10⁸ cfu/g and 5.1×10⁸ cfu/g compared to the group fed with general feed to 6.9×10⁸ cfu/g and 8.8×10⁸ cfu/g, respectively. In the cecum of broilers fed with the organic iron-containing feed, belonging to the negative control, the total CFU of lactic acid bacteria was 2.1×10⁸ cfu/g, which was lower than that of the general diet group.

### 3-2. 2nd Broiler Chicken

As in 3-1, a dietary test was performed for a total of 32 days using 240 1-day-old broiler chickens (ROSS 308) (New Materials Research Institute Test Farm, Deokso, Gyeonggi-do, Korea). 4 groups with 10 chickens per group, general feed (BD, basal diet, JoongAng Livestock broiler feed), general feed and 0.5 ppm of hemoP1 hemoprotein (BD+hemoprotein 0.5 ppm), general feed and 1 ppm of hemoP1 hemoprotein (BD+hemoprotein 1 ppm), and general feed and dried hemoP1 culture broth (dried powder obtained from the entire hemoP1 culture broth) 5 ppm (BD+hemoprotein culture 5 ppm) were formed and the dietary test was performed in 6 replicates. During this period, the body weight gain and feed intake of the broiler were measured, and a cecum was excised and plated on nutrient broth (NB) and MRS medium to measure the CFU of lactic acid bacteria.

TABLE 3

| | | | Lactic acid | | |
|---|---|---|---|---|---|
| | FCR | FCR | bacteria in | | |
| | (feed intake/ | ratio | the cecum | Body weight | Body weight |
| Diet | weight gain) | (%) | (10⁸ cfu/g) | gain (g) | gain rate (%) |
| BD | 1.58 ± 0.06 | 100.0 | 0.5 | 1514.1 ± 69.4 | 100 |
| BD + Hemoprotein | 1.56 ± 0.06 | 101.3 | 6.83 | 1435.9 ± 52.7 | 94.8 |
| 0.5 ppm | | | | | |
| BD + Hemoprotein | 1.56 ± 0.07 | 101.3 | 1.79 | 1454.1 ± 31.8 | 96.1 |
| 1 ppm | | | | | |
| BD + Hemoprotein | 1.55 ± 0.08 | 101.9 | 7.25 | 1473.2 ± 58.3 | 97.3 |
| Culture Broth | | | | | |
| 5 ppm | | | | | |

It was found that when 0.5 ppm or 1 ppm of hemoP1 hemoprotein or 5 ppm of dried hemoP1 culture broth was fed with general feed, body weight decreased by 5.2%, 3.9%, and 2.7%, respectively, compared to broilers fed only with general feed, and the CFU of lactic acid bacteria in the cecum in the groups were 6.83*10⁸ cfu/g, 1.79*10⁸ cfu/g, and 7.25*10⁸ cfu/g, respectively, which are 13.66 times, 3.58 times, and 14.5 times higher than the group fed with general feed.

### 3-3. Laying Hens

The experiment was conducted for 8 weeks (Feb. 24, 2020˜Apr. 20, 2020) using 42 laying hens aged 78 weeks. A total of two groups were formed, a group fed with general feed (BD) and a group fed with general feed and 1 ppm hemoprotein (BD+1 ppm hemoprotein). During this period, the eggs laid were collected and their weights were measured, and after the experiment was over, the cecum was excised and smeared on MRS medium, and the CFU of lactic acid bacteria was measured.

TABLE 4

| | Egg weight | Egg weight | Lactic acid bacteria in |
|---|---|---|---|
| Diet | (g) | ratio (%) | cecum (10⁸ cfu/g) |
| BD | 64 ± 2.2 | 100.0 | 3.3 |
| BD + Hemoprotein | 62 ± 1.4 | 96.8 | 4.5 |
| 1 ppm | | | |

When fed with the general feed and 1 ppm of hemoprotein, the weight of eggs decreased by 3.2% and the CFU of lactic acid bacteria in the cecum increased by 36% compared to the group fed with the general feed only.

### Example 4. Effect of Microbial Hemoprotein Extract on Body Weight Control and Intestinal Function in Obese Mice (1st Obese Mouse Experiment)

In this Example, the effects of the microbial hemoprotein extract on body weight control, that is, inhibition of body weight gain and intestinal function effect were studied in mice induced to be obese by a high fat diet (HFD).

The microbial hemoprotein extract was a hemoP sample prepared as described in Example 1. The microbial hemoprotein extract was mixed at 0.5 g/kg (HFD) and 5 g/kg (HFD) in 60% HFD and fed ad libitum.

The normal control group was given a basic diet, an AlN 93G diet ad libitum, and the obese control group was given a 60% HFD (60% fat per kcal, Research Diets Inc., New Brunswick, NJ, USA) ad libitum (Table 5).

Five-week-old male C57BL/6 mice purchased from Hana Bio (Korea) were used after they were confirmed as healthy following inspection and acclimation for 7 days at Dongnam Chemical Research Institute Animal Center. During the feeding, the lighting time was set to a 12-hour cycle, and food and water were provided ad libitum. This experiment was conducted in accordance with the policies and regulations of the Dongnam Medical Chemical Research Institute Animal Experiment Ethics Committee (No. SEMI-22-001, Institutional Animal Care and Use Committee).

### 4-1. Induction of Obesity in Mice

To induce obesity, test animals (n=20), excluding the normal group (N, n=5), were fed with a high fat diet (HFD) with the composition shown in Table 5 below for 8 weeks. The normal group (N) was fed with the AlN 93G diet ad libitum, which is a general feed.

TABLE 5

HFD (D12492)

| Ingredient | g | kcal |
|---|---|---|
| Casein, 30 mesh | 200 | 800 |
| L-cysteine | 3 | 12 |
| Corn Starch | 0 | 0 |
| Maltodextrin 10 | 125 | 500 |
| Sucrose | 68.8 | 275 |
| Cellulose, BW 200 | 50 | 0 |
| Soybean oil | 25 | 225 |
| Lard | 245 | 2205 |
| Mineral Mix S10026 | 10 | 0 |
| Dicalcium Phosphate | 13 | 0 |
| Calcium Carbonate | 5.5 | 0 |
| Potassium Citrate | 16.5 | 0 |
| Monohydrate | | |
| Vitamin Mix V10001 | 10 | 40 |
| Choline Bitartrate | 2 | 0 |
| FD&C Red Dye #40 | 0 | 0 |
| FD&C Blue Dye #1 | 0.05 | 0 |
| FD&C Yellow Dye #40 | 0 | 0 |
| Total | 773.85 | 4057 |

After inducing obesity, the animals were divided into a group fed with HFD alone (obesity control group, C), a group fed with HFD and hemoP 0.5 g/kg (0.05%) (SL), and a group fed with HFD and hemoP 5 g/kg (0.5%) (SH) so that the average body weight and standard deviation between groups were uniform (5 animals per group). During the period of high fat diet and hemoP feeding, the body weight, feed intake, and water consumption of the test animals were measured every three days.

### 4-2. Sacrifice and Tissue Extraction

After completion of the experiment, the test animals were anesthetized with CO₂ and sacrificed, and to compare the fat weight of each group, the peri-epididymal fat, the peri-renal fat, retroperitoneal fat, and mesenteric fat were extracted and weighed.

### 4-3. Results

### (1) Obesity Induction

As described in 4-1, 20 mice (obese group) fed with a high fat diet for 8 weeks continued to gain weight while being fed with a high fat diet, and the average body weight exceeded 40 g after 8 weeks. Compared to the normal group (N) fed with a normal diet, the weight gain of the obese group was approximately 172%, which showed that obesity was induced.

### (2) Effect of Hemoprotein Extract.

### —Body Weight

An obese mouse model was produced using the high fat diet as confirmed in (1), and then the model was fed with a diet mixed with hemoprotein extract for 2 weeks. Changes in body weight of mice were monitored during the feeding with a diet mixed with hemoprotein extract. Table 6 shows the weight measurement results. Each value is expressed as the mean±standard deviation of five measurements. The mixed feeding groups SL and SH were fed with a high fat diet mixed with 0.5 g/kg and 5 g/kg of hemoprotein extract hemoP, respectively.

TABLE 6

| | Body weight (g) | | | |
|---|---|---|---|---|
| | Normal control | Obese control | Mixed feeding | Mixed feeding |
| Week | group (N) | group (C) | group (SL) | group (SH) |
| Initial | 26.799 ± 0.315 | 41.866 ± 1.769 | 41.720 ± 0.380 | 41.795 ± 2.363 |
| Week 1 | 27.346 ± 0.341 | 43.911 ± 1.292 | 41.570 ± 0.404 | 41.943 ± 2.310 |
| Week 2 | 27.760 ± 0.291 | 46.722 ± 1.099 | 43.771 ± 0.299 | 44.324 ± 2.146 |
| Change in body | 0.961 | 4.856 | 2.051 | 2.529 |
| weight | | | | |

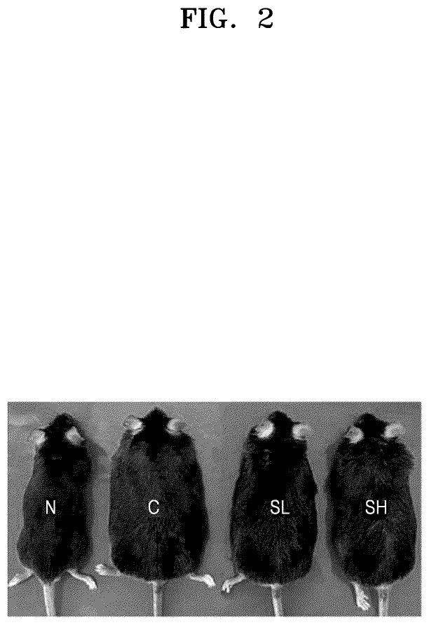

FIG. 2 shows mice in the normal control group (N), the obese control group (C), the 0.5 g/kg hemoprotein extract mixed feeding group (SL), and the 5 g/kg hemoprotein extract mixed feeding group (SH) after 2 weeks of feeding. Compared to the obese control group (C), the SL and SH groups mix fed with hemoprotein extract had significantly lower weight gain and were slimmer in appearance.

### —Adipose Tissue Weight

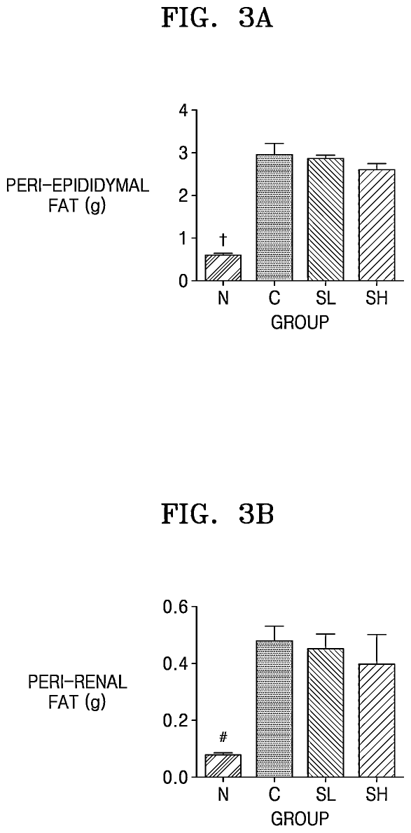

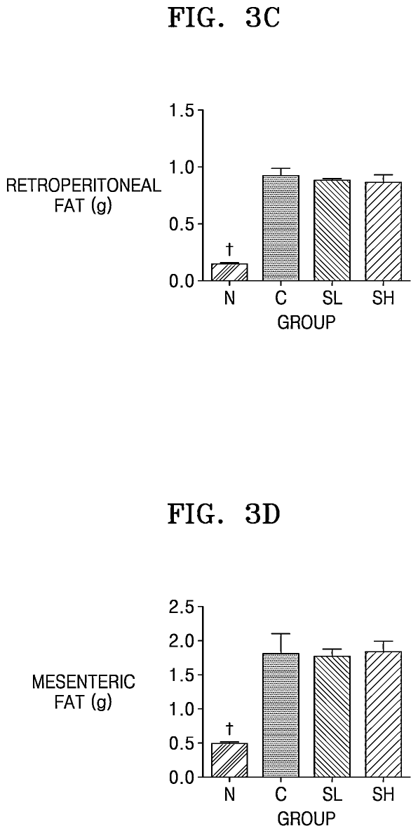

Adipose tissue weight in all groups fed with a high fat diet increased compared to the normal control group. Peri-epididymal, peri-renal, retroperitoneal, and mesenteric adipose tissue weights were all reduced in the normal control group (N) compared to the obese control group (C), and the SL and SH groups mix fed with a hemoprotein extract also showed reductions in the fat weights compared to the obese control group. The results are shown in FIG. 3. (A) to (D) of FIG. 3 show the weight of peri-epididymal fat, peri-renal fat, retroperitoneal fat, and mesenteric adipose tissue, respectively.

### —Adipose Tissue Weight to Body Weight Ratio

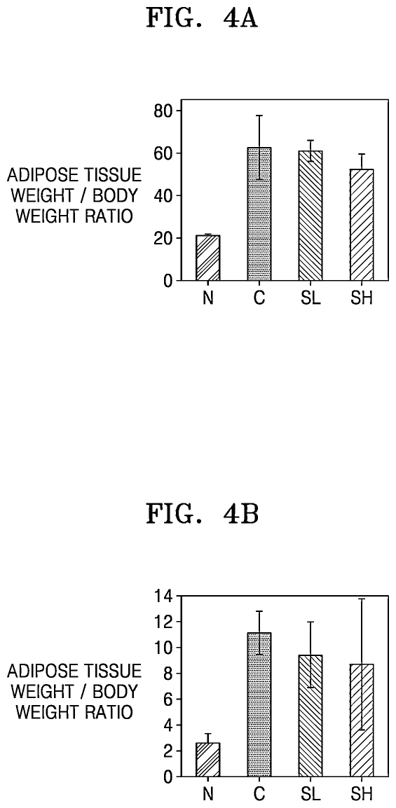

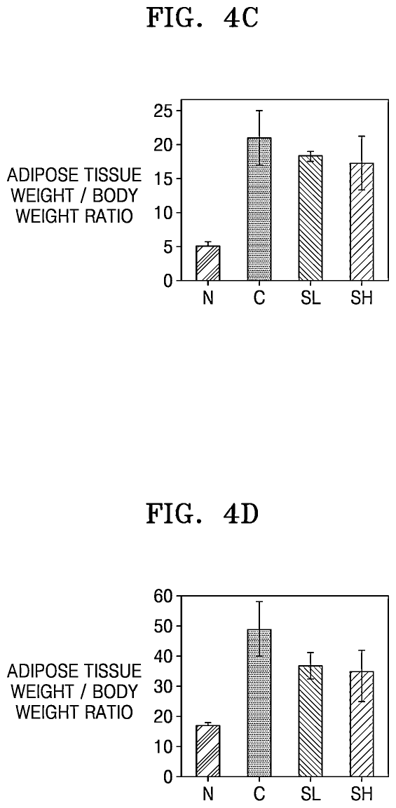

The ratio of adipose tissue weight to body weight was measured for each group of mice. The results are shown in FIG. 4. The ratio of adipose tissue weight to body weight in the normal control group was significantly reduced in all peri-epididymal fat, renal fat, retroperitoneal fat, and mesenteric fat compared to the obese control group. In addition, the SL and SH groups mix fed with hemoprotein extract also showed a decrease in the ratio of adipose tissue weight to body weight compared to the obese control group.

### —Proportion of Akkermansia Spp. in Intestinal Flora

Microbes belonging to the genus Akkermansia are facultative anaerobes, and their proportion in the intestinal flora generally decreases with obesity and increases with normal body weight, as reported in a recent study (https://microbiome.chunlab.com/wiki/akkermansia-muciniphila/).

Immediately before sacrifice of the test animals, feces were collected from the mouse cages of the obese control group, mixed feeding group SL, and SH, and the amount of intestinal microbes belonging to the genus Akkermansia in the intestinal flora was measured based on 16S rRNA sequence (CJ Life Science Microbiome Analysis Laboratory, Suwon, Korea). The results are shown in Table 7.

TABLE 7
Proportion of microorganisms in the genus Akkermansia (%)

| Obese control | Mixed feeding group | Mixed feeding group |
|---|---|---|
| group (C) | (SL) | (SH) |
| 4.24 | 15.16 | 11.33 |

The proportion of the microbes of the genus Akkermansia was only 4.24% in the obese control group, while the proportion was higher than that in the mixed feeding groups with 15.16% and 11.33, showing a tendency to be inversely proportional to obesity.

### Example 5. Effect of Microbial Hemoprotein Extract on Body Weight Control in Obese Mice (2nd Obese Mouse Experiment)

By using the same method as in Example 4, a second experiment was conducted in order to reconfirm the effect of the microbial hemoprotein extract on the weight control, that is, the inhibition of weight gain and the intestinal function in the mice in which obesity was induced by the high fat diet (HFD). After sacrifice, subcutaneous fat was additionally extracted from the extracted adipose tissue and its weight was measured. The results are shown in Table 8 and Table 9. Table 8 and Table 9 show the change in body weight for 10 days and the weight of adipose tissue extracted after sacrifice in mice induced to obesity and fed ad libitum a high fat diet mixed with 0.05% and 0.5% by weight of hemoprotein extract, respectively.

TABLE 8

| | Body weight (g) | | | |
|---|---|---|---|---|
| | Normal control | Obese control | Mixed feeding | Mixed feeding |
| Week | group (N) | group (C) | group (SL) | group (SH) |
| Initial | 24.801 ± 1.033 | 34.906 ± 2.228 | 34.723 ± 2.563 | 34.648 ± 2.338 |
| Day 5 | 25.466 ± 0.684 | 36.359 ± 2.355 | 35.477 ± 2.732 | 35.927 ± 2.797 |
| Day 10 | 25.341 ± 0.934 | 37.840 ± 2.488 | 36.469 ± 2.943 | 37.401 ± 2.862 |
| Change in body | 0.540 | 2.934 | 1.746 | 2.753 |
| weight | | | | |

TABLE 9

| | Adipose tissue weight (g) | | | |
|---|---|---|---|---|
| | Normal control | Obese control | Mixed feeding | Mixed feeding |
| Tissue | group (N) | group (C) | group (SL) | group (SH) |
| Epididymal fat | 0.491 ± 0.084 | 2.581 ± 0.347 | 2.290 ± 0.364 | 2.282 ± 0.392 |
| Renal Fat | 0.056 ± 0.015 | 0.238 ± 0.052 | 0.203 ± 0.054 | 0.230 ± 0.045 |
| Mesenteric Fat | 0.399 ± 0.040 | 1.078 ± 0.130 | 0.922 ± 0.521 | 0.984 ± 0.226 |
| Retroabdominal | 0.110 ± 0.023 | 0.751 ± 0.144 | 0.737 ± 0.178 | 0.726 ± 0.102 |
| fat | | | | |
| Subcutaneous fat | 0.297 ± 0.045 | 2.373 ± 0.571 | 1.313 ± 0.305 | 1.596 ± 0.390 |

All three groups fed with the high fat diet (C, SL, SH) showed higher body weight and adipose tissue weight than the normal control group (N). Among the high fat diet groups, the body weight and the weights of all adipose tissues in the two group mix fed with hemoprotein (SL and SH) were lower than those in the obese control group (C). Therefore, the effect of weight loss and adipose tissue reduction in obese mice by hemoprotein supply was reconfirmed.

### Example 6. Effect of Microbial Hemoprotein Extract on Intestinal Function in Pet Dogs Via Discretionary Feeding Thereof

In order to investigate the effect of microbial hemoprotein extract on intestinal function in dogs, microbial hemoprotein extract containing snack products (trade name: Bio-Treat; feed registration number: Seoul-28372; type: mixed feed ingredients; ingredient amount: crude protein 10.1%, crude fat 5%, crude fiber 0.7%, crude ash 2.3%, moisture 18%; Name of ingredients: yellow squash 20%, sweet pumpkin 14.8%, duck 10%, carrot 10%, brown rice 5%, salmon 5%, mussel 5%, cabbage 4%, sweet potato 5%, broccoli 3%, coconut 3%, tapioca starch 10%, glycerin 5%, hemoprotein [Corynebacterium fermentation product] 0.2%; Manufacturer: Hytek Korea; Distributor: HemoLab) were provided along with normal feed so that each dog owner fed a dog with normal feed or normal feed and the microbial hemoprotein extract containing snack products at the discretion of each dog owner, and fecal samples were collected from each dog owner's dog for 2 weeks.

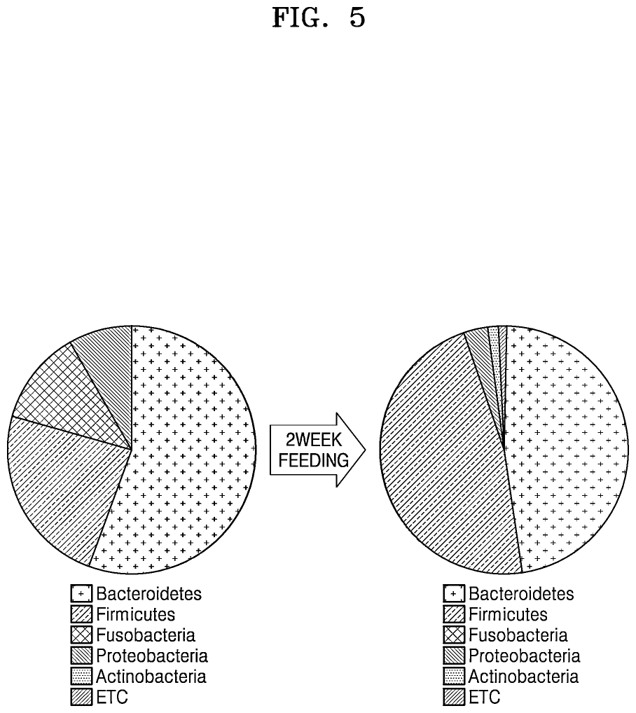

Fecal samples collected before providing the snacks and after feeding one bag of the snack product (total weight: 100 g) were obtained and the bacterial flora thereof was examined. The distribution of intestinal flora in fecal samples was analyzed based on 16S rRNA sequence (CJ Life Science Microbiome Analysis Laboratory, Suwon, Korea). The results are shown in FIG. 5.

It was found that compared to the fecal samples collected before providing the hemoprotein extract containing snack product, the fecal samples collected after providing the snack product showed a significant increase in the proportion of the Firmicutes, a phylum dominated by the beneficial bacteria, Lactic acid bacteria, and a decrease in the proportion of proteobacteria, a phylum dominated by E. coli, etc. Even with the consumption of the hemoprotein extract through discretionary feeding, the intestinal flora of the pet dogs was found to have improved in the direction of increasing the proportion of beneficial bacteria.

These results show that microbial hemoprotein or dried microbial culture broth having high microbial hemoprotein content has a positive effect on the intestinal function and adipose tissue weight reduction effect.

1-7. (canceled)

8. A method of preventing or ameliorating obesity, comprising administering a microbial hemoprotein extract to a subject in need thereof.

9. The method of claim 8, wherein the microbial hemoprotein extract is in a form of microbial culture broth obtained by culturing a heme iron-producing microorganism, microbial biomass isolated therefrom, or a hemoprotein isolated and purified therefrom.

10. The method of claim 9, wherein the microorganism is a non-genetically modified microorganism selected through adaptive evolution for growth rate and heme iron productivity to have a higher hemoprotein content than a parent strain.

11. The method of claim 9, wherein the microbial hemoprotein extract is obtained by a method comprising recovering microbial biomass from the microbial culture broth, resuspending and disrupting the recovered microbial biomass, and centrifuging or drying the resulting suspension.

12. The method of claim 8, wherein the administering the microbial hemoprotein extract ameliorates obesity by improving gut health.

13. The method of claim 8, wherein the administering the microbial hemoprotein extract ameliorates obesity by reducing a fat-to-body weight ratio.

14. The methodπ of claim 8, wherein the administering the microbial hemoprotein extract increases the proportion of Akkermansia spp. in intestinal flora.

15. The method of claim 8, wherein the administering the microbial hemoprotein extract ameliorates obesity by decreasing body weight gain.

16. The method of claim 8, wherein the microbial hemoprotein extract is administered as feed, food, or health supplement.
